(19) United States
(12) Patent Application Publication
(10) Pub. No.: US 2013/0041434 A1
(43) Pub. Date: Feb. 14, 2013

(54) PAD FOR THERMOTHERAPHY

(71) Applicants: Doo Hyeb YOUN, Daejeon (KR); Sung-Yool Choi, Daejeon (KR); HongKyw Choi, Busan (KR); Jongyun Kim, Daejeon (KR)

(72) Inventors: Doo Hyeb YOUN, Daejeon (KR); Sung-Yool Choi, Daejeon (KR); HongKyw Choi, Busan (KR); Jongyun Kim, Daejeon (KR)

(73) Assignee: Electronics and Telecommunicatios Research Institute, Daejeon (KR)

(21) Appl. No.: 13/571,309

(22) Filed: Aug. 9, 2012

(30) Foreign Application Priority Data
Aug. 11, 2011 (KR) 10-2011-0080275

(51) Int. Cl. A61N 5/06

(52) U.S. Cl. 607/90

(57) ABSTRACT

A pad for thermotherapy includes: a stretchable and flexible substrate; an electrode pattern positioned over the stretchable and flexible substrate, and including a plurality of light source electrodes and a linear electrode connecting the light source electrodes; light sources positioned over the electrode pattern; and a power supply unit for supplying power to the light source, wherein the linear electrode is formed longer than intervals between neighboring light source electrodes and separated from the substrate.

### CROSS-REFERENCE TO RELATED APPLICATION

This application claims priority to and the benefit of Korean Patent Application No. 10-2011-0080275 filed in the Korean Intellectual Property Office on Aug. 11, 2011, the entire contents of which are incorporated herein by reference.

### BACKGROUND OF THE INVENTION

(a) Field of the Invention

The present invention relates to a pad for thermotherapy, and more particularly, to a pad for thermotherapy which is bendable according to the shape of a body.

(b) Description of the Related Art

Along with the rise of income levels and advances of civilization, there has been growing concern about beauty, and many studies aimed at improving skin are underway.

However, laser-based therapies or care for treating or improving skin diseases like wounds, acne, atopy, etc., are expensive, and cause a lot of inconvenience such as pain during treatment and wounds after treatment.

Accordingly, there is ongoing research on skin disease treatment using an LED, which allows easy selection of wavelengths of light, such as infrared light or ultraviolet light, as needed, offers low prices, causes less pain, and is expected to have therapeutic effects.

However, LED lights require flowing a large amount of electric current in order to get desired light and raise therapeutic effects, because 70% or more of incoming current is transformed into heat and lost, and is placed far from a treatment site.

Also, there is a problem that generated heat has effects on the output and wavelength of a therapeutic apparatus, thus reducing therapeutic efficiency.

### SUMMARY OF THE INVENTION

The present invention has been made in an effort to provide a pad for thermotherapy which reduces heat generation caused by electric current and achieves a stable output and wavelength.

An exemplary embodiment of the present invention provides a pad for thermotherapy including: a substrate; an electrode pattern positioned over the substrate, and including a plurality of light source electrodes and a linear electrode connecting the light source electrodes; light sources positioned over the electrode pattern; and a power supply unit for supplying power to the light sources, wherein the linear electrode is formed longer than intervals between neighboring light source electrodes and is separated from the substrate.

The pad may further include a buffer layer, and the light source electrodes may be positioned over the buffer layer.

The buffer layer may be made of a thermal conductive elastic rubber.

The buffer layer may comprise a thermal conductive polymer or a complex of polymer and metal nano-particles, in which the thermal conductivity is more than 3 W/mK. The buffer layer may be stretchable and flexible.

The substrate may be stretchable and flexible, and the substrate may be made of graphene.

The light sources may be LEDs that emit visible light, infrared light, or ultraviolet light.

The buffer layer and the electrode pattern may be formed by near-field electrospinning.

An exemplary embodiment of the present invention provides a pad for thermotherapy including: a band-shaped conductive substrate; an electrode positioned over the substrate; a fixing portion positioned over the electrode; a plurality of light sources inserted into the fixing portion and electrically connected to the electrode; and a power supply unit for supplying power to the light sources.

The light sources may emit visible light, infrared light, or ultraviolet light, and the electrode may be longitudinally formed in a band shape.

The pad may further include an insulating layer positioned over the fixing portion, and the electrode and the light sources may be electrically connected via through holes formed in the insulating layer.

The fixing portion and the insulating layer may be formed integrally with each other.

The band-shaped conductive substrate or the electrode may be stretchable and flexible.

According to the present invention, a graphene sheet can be used to place the pad for thermotherapy into contact with a treatment site and transmit light. Accordingly, it is possible to provide an optical output device which has a stable output and wavelength because heat generation, caused by a current increase made for light transmission to a treatment site, can be reduced.

### BRIEF DESCRIPTION OF THE DRAWINGS

FIG. 1 is a layout chart of a pad for thermotherapy according to an exemplary embodiment of the present invention.

FIG. 2 is a cross-sectional view taken along line II-II of FIG. 1.

FIG. 3 is a photograph showing an application example of a pad for thermotherapy according to an exemplary embodiment of the present invention which is attached to a human body.

FIGS. 4 and 5 are cross-sectional views sequentially showing a method for manufacturing a pad for thermotherapy according to an exemplary embodiment of the present invention.

FIG. 6 is a cross-sectional view of a pad for thermotherapy according to another exemplary embodiment of the present invention.

### DETAILED DESCRIPTION OF THE EMBODIMENTS

The present invention will be described more fully hereinafter with reference to the accompanying drawings, in which exemplary embodiments of the invention are shown. As those skilled in the art would realize, the described embodiments may be modified in various different ways, all without departing from the spirit or scope of the present invention.

In the drawings, the thickness of layers, films, panels, regions, etc., are exaggerated for clarity. Like reference numerals designate like elements throughout the specification. It will be understood that when an element such as a layer, film, region, or substrate is referred to as being “on” another element, it can be directly on the other element or intervening elements may also be present. In contrast, when an element is referred to as being “directly on” another element, there are no intervening elements present.

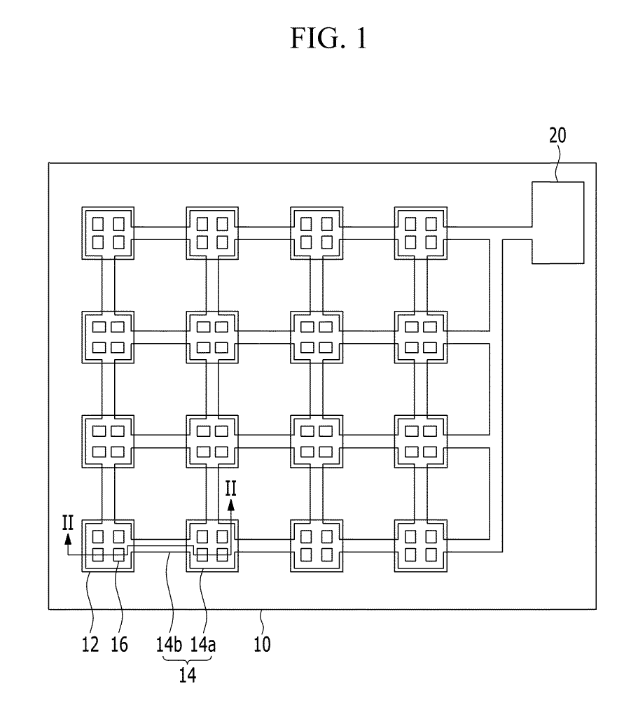

Hereinafter, a pad for thermotherapy according to an exemplary embodiment of the present invention will be described in detail with reference to FIGS. 1 to 5.

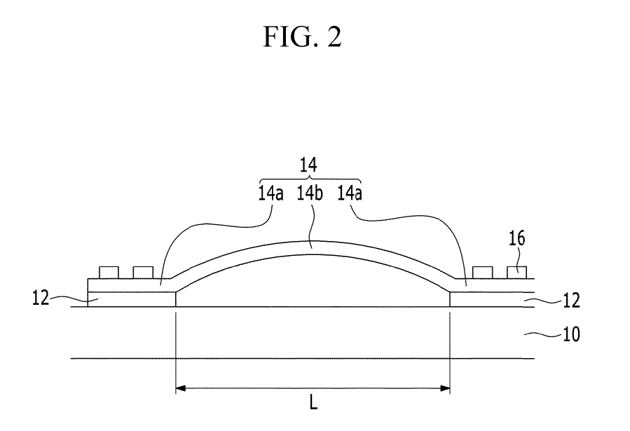

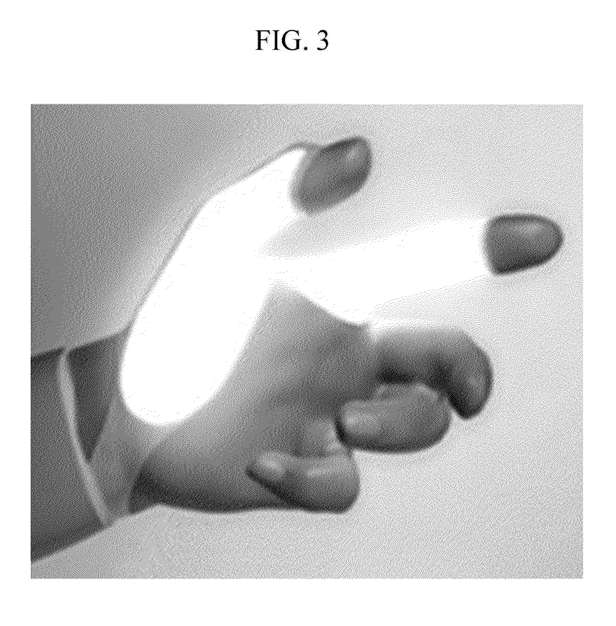

FIG. 1 is a layout chart of a pad for thermotherapy according to an exemplary embodiment of the present invention, FIG. 2 is a cross-sectional view taken along line II-II of FIG. 1, and FIG. 3 is a photograph showing an application example of a pad for thermotherapy according to an exemplary embodiment of the present invention which is attached to a human body.

As shown in FIGS. 1 and 2, the pad for thermotherapy according to the present invention includes a substrate 10, a buffer layer 12, an electrode pattern 14 positioned over the buffer layer 12, and light sources 16 positioned over the electrode pattern 14.

The substrate 10 may be made of a stretchable material which is bendable or expandable, for example, a graphene sheet.

The graphene sheet indicates graphene in the form of a film derived from polycyclic aromatic molecules in which a plurality of carbon atoms are covalently bound to each other. The covalently bound carbon atoms form repeating units that comprise 6-membered rings, but can also form 5-membered rings and/or 7-membered rings. Accordingly, in the graphene, it appears as if the covalently bound carbon atoms (usually an sp2 bond) form a single layer. The graphene sheet may have various structures and the structure may vary according to the amount of the 5-membered rings and/or the 7-membered rings. The graphene sheet may have not only a single layer of graphene, but also a plurality of layers up to a thickness of 100 nm. Generally, the side ends of the graphene are saturated with hydrogen atoms.

One property of the graphene sheet is that electrons flow therein as if the mass of the electrons is zero, which means that electrons flow at the velocity of light in vacuum. The electron mobility of graphene sheets is about 10,000 to 1,000,000 cm²/Vs.

In addition, graphene sheets which make sheet contact with each other have a far lower contact resistance value compared to carbon nanotubes which make point contact with each other. Problems caused by surface roughness can be prevented since the graphene sheet can have a very thin thickness, and the graphene sheet is economically prepared since it can be simply separated from inexpensive graphite.

The buffer layer 12 is for compensating for differences in elastic modulus between the substrate 10 and the electrode pattern 14 and for separating part of the electrode pattern 14 from the substrate 10, and may be made of an insulating elastic rubber with a high elastic modulus.

The electrode pattern 14 includes a plurality of light source electrodes 14a over which the light sources 16 are positioned and a linear electrode 14b connecting the plurality of light source electrodes 14a. The light source electrodes 14a are portions where the light sources 16 are positioned, and may have a greater width than the linear electrode 14b and have a polygonal shape such as a circular or rectangular shape.

The light source electrodes 14a are positioned over the buffer layer 12, and the linear electrode 14b electrically connects between the light source electrodes 14a, are longer than the intervals L between the light source electrodes 14a, and are separated from the substrate 12.

That is, the linear electrode 14b, if it has the same length as the intervals between the light source electrodes 14a, may be broken when the substrate 10 is stretched. On the other hand, if the linear electrode 14b is longer than the intervals between the light source electrodes 14a, as in the exemplary embodiment of the present invention, the linear electrode 14b is not broken because it is also stretched as the substrate 10 is stretched. Accordingly, electric current is seamlessly transmitted to the light sources 16 even if the substrate 10 is stretched.

The electrode pattern 14 may be formed by a near-field electrospinning method by using a graphene oxide solution blended to have an elastic modulus and viscosity of 2000 Pa or more, a solution of single-wall carbon nanotubes, and a solution containing fine particles of conductive metal particles.

The light sources 16 may be LEDs, and are electrically connected with the light source electrodes 14a. The light sources 16 may emit wavelengths of such light as visible light, infrared light, or ultraviolet light according to therapeutic purpose.

Electric power supplied from the power supply unit 20 is transmitted to the light source electrodes 14a through the linear electrode 14b, and causes an equal amount of electric current to flow to the light sources 16 positioned over the entire substrate 10. The power supply unit 20 may supply electric power by wires, such as through an adapter, or by a battery mounted on the substrate. The power supply unit 20 may further include a switching device (not shown) for applying electric power as required.

As in the exemplary embodiment of the present invention, a pad for thermotherapy is formed by forming light sources on a stretchable substrate, and an adhesive bond pad or a fixing portion with a button is formed on the substrate of the pad for thermotherapy so that it can be attached like a patch to a treatment site such as a wrist, a knee, etc., as shown in FIG. 3 to perform thermotherapy.

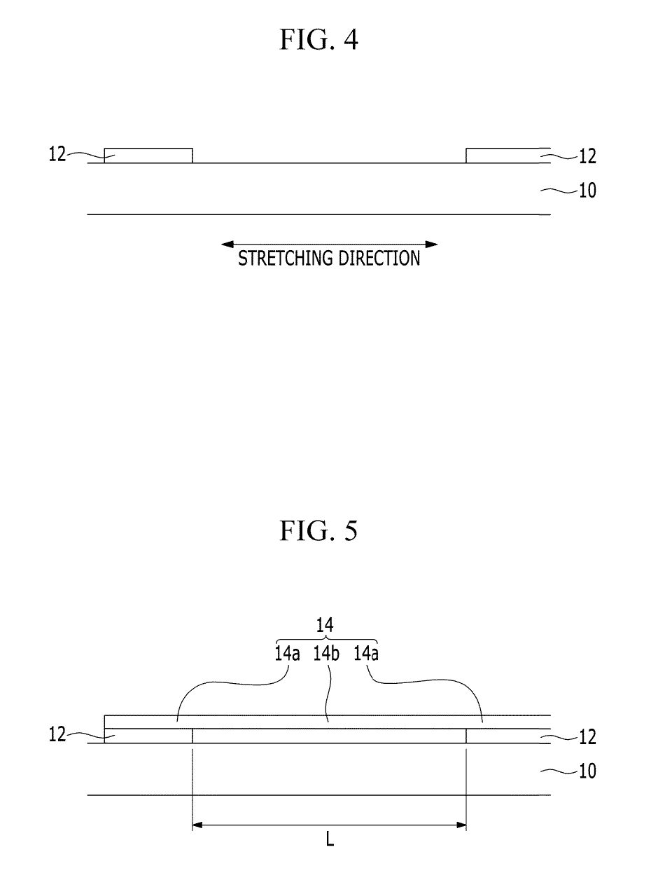

By firmly attaching an adhesive pad with light sources arranged therein for the treatment of skin diseases to the skin, as shown in FIG. 4, the intensity of the light sources can be increased by more than five times compared to conventional light sources which are placed far from the skin and irradiate light from a far distance. Accordingly, there is no need for a large inflow of electric current to increase the intensity of the light sources. Thus, sufficient therapeutic effects can be obtained by only a small inflow of electric current, and less heat is generated, thereby preventing changes in the intensity and wavelength of the light sources caused by an increased inflow of electric current.

By attaching the adhesive pad to the body requiring thermotherapy and transmitting light thereto, as described above, light having the same intensity is irradiated onto the treatment site by a smaller amount of electric current than that used to irradiate light from a far distance from the treatment site. Accordingly, electric current does not need to be increased in order to increase the intensity of light, thereby minimizing heat generation.

A method for manufacturing the above-described pad for thermotherapy will be described in detail with reference to FIGS. 4 and 5 and the above-explained FIG. 2.

FIGS. 4 and 5 are cross-sectional views sequentially showing a method for manufacturing a pad for thermotherapy according to an exemplary embodiment of the present invention.

First, as shown in FIG. 4, a stretchable, conductive substrate 10 is prepared, and a buffer layer 12 is formed on the substrate 10.

Afterwards, as shown in FIG. 5, the substrate 10 is stretched along with the buffer layer 12, and then the electrode pattern 14 is formed on the substrate 10 by near-field electrospinning. At this point, the light source electrodes 14a are positioned over the buffer layer 12, and the linear electrode 14b does not overlap with the buffer layer 12 and looks as if it is floating on the substrate 10.

In the near-field electrospinning, a conductive solution with a mixture of conductive materials such as carbon nanotubes is sprayed via a spray nozzle when a voltage is applied.

The conductive solution may be sprayed by the use of a spray nozzle and a ground collector corresponding to the spray nozzle and forming an electric field. As the conductive solution is discharged from the spray nozzle, tiny droplets are formed at the tip of the spray nozzle, and the surface tension for keeping the original shape of the droplets and an electric field applied between the spray nozzle and the conductive ground collector are in equilibrium. Afterwards, if the strength of the voltage applied to the spray nozzle is increased, the equilibrium between the surface tension and the applied voltage is destroyed as the strength of the voltage exceeds a critical potential, and linear fluid columns start to be ejected from the round droplets. As this fluid is electrically charged, conduction current flows to supplement electric charges equivalent to the amount of ejected electric charges. Accordingly, the droplets sprayed from the tip of the nozzle in the form of an electrically charged solution are not dispersed by their surface tension, but stick to the ground collector as soon as they are sprayed by electrostatic repulsion against the voltage applied to an injection needle.

In the near-field electrospinning, a micropattern can be formed by adjusting the size of the nozzle, the distance between the nozzle and the ground collector, and so on.

Next, as shown in FIG. 2, the stretching force is removed from the stretched substrate 10 to avoid stretching of the substrate 10, and then an LED is formed on the electrode pattern 14. The LED may be formed directly on the light source electrodes 14a.

Since the force applied to the stretched substrate 10 is released and the substrate 10 is no longer stretched, the linear electrode 14b of the electrode pattern 14 formed in the stretched state is bent. That is, the light source electrodes 14a are positioned over the buffer layer 12, while the linear electrode 14b is positioned between neighboring light source electrodes 14a and does not overlap with the buffer layer 12 and looks as if it is floating. Accordingly, if the stretching force is no longer applied to the stretched substrate 10 and the substrate 10 returns to its original size, the intervals between the light source electrodes 14a become narrower and the linear electrode 14b is bent as much as the light source electrodes 14a become closer.

By forming the linear electrode 14b to be bendable as in the exemplary embodiment of the present invention, even if a person having the pad for thermotherapy attached to the body moves their body, the pad for thermotherapy is stretched according to the motion of the body and the linear electrode 14b is also stretched. This prevents breaking of the linear electrode 14b.

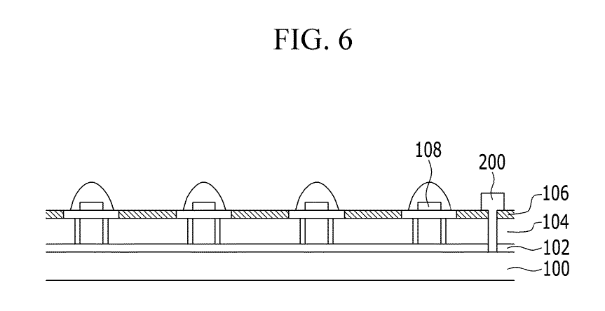

FIG. 6 is a cross-sectional view of a pad for thermotherapy according to another exemplary embodiment of the present invention.

The pad for thermotherapy of FIG. 6 includes a substrate 100, an electrode 102, an insulating layer 104 positioned over the electrode 102, a fixing portion 106 positioned over the insulating layer 104, and light sources 108 inserted into the fixing portion 106.

Unlike the exemplary embodiment of FIGS. 1 and 2, the substrate 100 is not stretchable but bendable, and may surround the treatment site such as an arm, leg, etc., in a band shape.

An electrode may be formed on the substrate 100 by forming a low-resistance metal layer of copper or the like and patterning the metal layer, or by near-field electrospinning.

The fixing portion 106 is for fixing the light sources 108, and may be formed integrally with the insulating layer 104 and fix the light sources 108 by inserting the light sources 108 into holes formed in the fixing portion 106.

While the LED of FIGS. 1 and 2 is integrated directly on the electrode, the LED of FIG. 6 comes in the form of a module, and a hole is formed in the fixing portion so as to correspond to the size of the module.

The light sources 106 are electrically connected to the electrode 102 via through holes formed in the insulating layer 104.

Although the exemplary embodiment of the present invention has been described with respect to the fixing portion 106 and the insulating layer 104 formed as different layers, the fixing portion 106 and the insulating layer 104 may be formed integrally with each other.

When power is applied to one side of the electrode 102 from the power supply unit 200 positioned on one side of the substrate 100, electric current is sequentially applied to the light sources 106 above the substrate 100 through the electrode 102 formed along the substrate 100.

By forming the pad for thermotherapy in the shape of a band as described above, the pad can be attached to a treatment site such as an arm, leg, etc. to transmit light. Thus, there is no need to increase electric current to increase light efficiency. Accordingly, heat generation caused by a current increase can be minimized.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

### <Description of Symbols>

| | |
|---|---|
| 10, 100: substrate | 12: buffer layer |
| 14: electrode pattern | 14a: light source electrode |
| 14b: linear electrode | 16, 108: light source |
| 102: electrode | 104: insulating layer |
| 106: fixing portion | |

What is claimed is:

1. A pad for thermotherapy comprising:
a substrate;
an electrode pattern positioned over the substrate, and including a plurality of light source electrodes and a linear electrode connecting the light source electrodes;
light sources positioned over the electrode pattern; and
a power supply unit for supplying power to the light sources,
wherein the linear electrode is formed longer than intervals between neighboring light source electrodes and is separated from the substrate.

2. The pad of claim 1, further comprising a buffer layer positioned over the substrate.

3. The pad of claim 2, wherein the light source electrodes are positioned over the buffer layer.

4. The pad of claim 3, wherein the buffer layer is made of a thermal conductive elastic rubber.

5. The pad of claim 2, wherein the buffer layer is formed by near-field electrospinning.

6. The pad of claim 2, wherein the buffer layer is stretchable and flexible.

7. The pad of claim 1, wherein the substrate is stretchable and flexible.

8. The pad of claim 7, wherein the substrate is made of graphene.

9. The pad of claim 1, wherein the light sources are LEDs that emit visible light, infrared light, or ultraviolet light.

10. The pad of claim 1, wherein the electrode pattern is formed by near-field electrospinning.

11. A pad for thermotherapy comprising:
a band-shaped conductive substrate;
an electrode positioned over the substrate;
a fixing portion positioned over the electrode;
a plurality of light sources inserted into the fixing portion and electrically connected to the electrode; and
a power supply unit for supplying power to the light sources.

12. The pad of claim 11, wherein the light sources emit visible light, infrared light, or ultraviolet light, and the electrode may be longitudinally formed in a band shape.

13. The pad of claim 11, wherein the electrode is longitudinally formed in a band shape.

14. The pad of claim 11, further comprising an insulating layer positioned over the fixing portion, and
the electrode and the light sources are electrically connected via through holes formed in the insulating layer.

15. The pad of claim 14, wherein the fixing portion and the insulating layer are formed integrally with each other.

16. The pad of claim 11, wherein the band-shaped conductive substrate is stretchable and flexible.

17. The pad of claim 11, wherein the electrode is stretchable and flexible.
